(19) United States
(12) Patent Application Publication
(10) Pub. No.: US 2011/0087288 A1
(43) Pub. Date: Apr. 14, 2011

(54) Surgical Fixation System and Related Methods

(76) Inventors: Tara Stevenson, San Diego, CA (US); Richard Mueller, Carlsbad, CA (US); Steven Vanni, N. Miami Beach, FL (US)

(21) Appl. No.: 12/739,248

(22) PCT Filed: Oct. 24, 2008
(86) PCT No.: PCT/US08/81227

§ 371 Date: Dec. 17, 2010

Related U.S. Application Data
(60) Provisional application No. 61/000,351, filed on Oct. 24, 2007.
(60) Provisional application No. 61/000,350, filed on Oct. 24, 2007.

(51) Int. Cl. A61B 17/70

(52) U.S. Cl. 606/250

(57) ABSTRACT

A surgical fixation system including a pair of spinal rods, an occipital fixation element (comprising either an occipital plate or a plurality of occipital anchors), a crosslink connector, and a plurality of anchor elements, including but not limited to friction-fit pedicle screws, favored-angle pedicle screws, and laminar hooks. Any or all of these elements may be made of a biologically inert material, preferably any metal customarily used for surgical devices, such as for example titanium or stainless steel. The surgical fixation system of the present invention is described herein for application to the posterior region of the human spine, for attachment to cervical and/or thoracic vertebrae, as well as the occiput portion of the skull.

### CROSS REFERENCES TO RELATED APPLICATIONS

The present application is an international patent application claiming benefit under 35 U.S.C. §119(e) from U.S. Provisional Application Ser. No. 61/000,350, filed on Oct. 24, 2007, and U.S. Provisional Application Ser. No. 61/000,351, filed on Oct. 24, 2007 the entire contents of which are hereby expressly incorporated by reference into this disclosure as if set forth fully herein.

### BACKGROUND OF THE INVENTION

I. Field of the Invention

The present invention relates generally to the field of spinal fixation devices, and more specifically to posterior cervical fixation assemblies for securing an orthopedic rod to a spine.

II. Background

The spinal column is a highly complex system of bones and connective tissues that provides support for the body and protects the delicate spinal cord and nerves. The spinal column includes a series of vertebral bodies stacked one atop the other, each vertebral body including an inner or central portion of relatively weak cancellous bone and an outer portion of relatively strong cortical bone. Situated between each vertebral body is an intervertebral disc that cushions and dampens compressive forces exerted upon the spinal column. A vertebral canal containing the spinal cord is located behind the vertebral bodies.

There are many types of spinal column disorders including scoliosis (abnormal lateral curvature of the spine), excess kyphosis (abnormal forward curvature of the spine), excess lordosis (abnormal backward curvature of the spine), spondylothesis (forward displacement of one vertebra over another), and other disorders caused by abnormalities, disease or trauma, such as ruptured or slipped discs, degenerative disc disease, fractured vertebra, and the like. Patients that suffer from such conditions usually experience extreme and debilitating pain, as well as diminished nerve function.

Surgical techniques commonly referred to as spinal fixation use surgical implants for fusing together and/or mechanically immobilizing two or more vertebral bodies of the spinal column. Spinal fixation may also be used to alter the alignment of adjacent vertebral bodies relative to one another so as to change the overall alignment of the spinal column. Such techniques have been used effectively to treat the above-described conditions and, in most cases, to relieve pain.

One spinal fixation technique involves immobilizing the spine using orthopedic stabilizing rods, commonly referred to as spine rods, which run generally parallel to the spine. This may be accomplished by exposing the spine posteriorly, and fastening bone screws to the pedicles of the vertebral bodies. The pedicle screws are generally placed two per vertebra and serve as anchor points for the spine rods. Clamping or coupling elements adapted for receiving a spine rod therethrough are then used to join the spine rods to the pedicle screws. The aligning influence of the spine rods forces the spinal column to conform to a more desirable shape. In certain instances, the spine rods may be bent to achieve the desired curvature of the spinal column.

There are many disadvantages associated with current spinal fixation devices. For example, many prior art bone fixation devices are less than optimal for capturing spine rods when the coupling elements must be rotated to extreme angles. With such devices, pivotal movement of the anchor portion is limited to an angle of generally no more than 40° (measured from vertical) in any direction. Surgeons have encountered considerable difficulty attempting to insert spinal fixation devices when the coupling elements are out of alignment with one another due to curvature of the spinal column and the different orientation of adjacent pedicles receiving screws. As a result, spine rods must often be bent in multiple planes in order to pass the rods through adjacent coupling elements. This may potentially weaken the overall assembly and results in longer operations and a greater likelihood of complications. Further problems may arise when applying an occipital plate due to the natural curvature of a patient's spine.

The present invention is directed at overcoming, or at least improving upon, the disadvantages of the prior art.

### SUMMARY OF THE INVENTION

The present invention accomplishes this goal by providing a surgical fixation system including a pair of spinal rods, an occipital fixation element (comprising either an occipital plate or a plurality of occipital anchors), a crosslink connector, and a plurality of anchor elements, including but not limited to friction-fit pedicle screws, favored-angle pedicle screws, and laminar hooks. Any or all of these elements may be made of a biologically inert material, preferably any metal customarily used for surgical devices, such as for example titanium or stainless steel. The surgical fixation system of the present invention is described herein for application to the posterior region of the human spine, for attachment to cervical and/or thoracic vertebrae, as well as the occiput portion of the skull. However, it should be noted that a surgical fixation system of the type described herein may find application to other parts of the body.

By way of example only, the occipital plate of the surgical fixation system comprises a generally flat body portion flanked by a pair of side-loading clamp elements, each dimensioned to receive one of the spinal rods. The body portion includes a plurality of apertures, each dimensioned to receive an anchor element such as an occipital screw. The clamp elements extend laterally (and generally opposite one another) from the body portion. Each clamp element comprises a first clamp portion and a second clamp portion. The first clamp portion is a generally flat extension of the bottom surface, while the second clamp portion is a curved element protruding generally perpendicularly out of the top surface such that the first and second clamp portions together form a generally U-shaped channel therebetween. The channel is dimensioned to receive at least a portion of the spinal rod, and first clamp portion includes a detent within channel in order to allow a “snap-fit” engagement between the clamp element and spinal rod. In order to further secure the rod within the clamp element, the second clamp portion includes an aperture dimensioned to receive a setscrew, which functions as a locking element to secure the spinal rod in place. In order to achieve this locking interaction, the setscrew threadedly engages the aperture such that the setscrew may be advanced toward the spinal rod until an angled surface located at the distal tip of the setscrew contacts the rod. In practice, the setscrew may be advanced to an extent such that the angled surface causes a slight deformation in the spinal rod, thereby preventing the rod from being expelled from the channel and effectively locking the spinal rod to the occipital plate. The apertures may be provided at an angle offset from the perpendicular extension of second clamp portion. Providing the apertures at an angle provides the occipital plate with an improvement in that the screw insertion becomes significantly less troublesome for surgeons to perform, due to the natural curvature of a patient's spine.

By way of example only, the crosslink connector is provided as a unitary member having a pair of opposing clamp portions separated by an elongated central portion. Each clamp portion includes curved extension forming a channel dimensioned to receive at least a portion of the spinal rod therein. The clamp portion further includes aperture dimensioned to threadedly receive a setscrew for locking the spinal rod within the channel. The aperture may be provided such that its longitudinal axis is medially offset at an angle relative to an axis extending perpendicularly from the longitudinal axis of the spinal rod. This disposition of the apertures is advantageous in that it allows for a more direct approach for inserting the setscrews.

The friction-fit polyaxial pedicle screw assembly includes a coupling element, an anchor element, a compression cap and a set screw.

By way of example only, the coupling element is generally cylindrical in shape with a proximal end and a distal end. The coupling element includes a passage extending axially therethrough from the proximal end to the distal end. At the distal end is an opening dimensioned to permit passage of the threaded portion of the anchor element, but not the head of the anchor element. The distal portion of the passage forms a seat for engaging the head, the seat being constructed so as to receive a partially spherically shaped region corresponding to the size and shape of the head of the anchor element. The seat is also constructed as having a diameter slightly smaller than that of the corresponding portion of the head of the anchor element. The coupling element further includes a pair of side extensions extending between the proximal end and distal end, and a U-shaped recess for receiving a least a portion of the spinal rod positioned between the side extensions. Within the passage toward the proximal end is a threaded region for threaded engagement with a locking member constructed as a nut or, preferably, a set screw. The coupling element may include one or more notches or detents on the side extensions for engaging an insertion device.

The anchor element is shown by way of example only as a screw including a distal tip for insertion into bone, a head at the proximal end thereof, and threaded shaft extending between distal tip and head. The head may include a recess adapted to cooperate with a driver used to sink the anchor element into bone. By way of example only, the recess is shown as a hex-head recess for receiving a hex-head driver. The head is preferably sized and shaped to pass through the passage of the coupling element until the head engages the seat. The head is generally spherical in shape and dimensioned to engage the seat. When the head engages the seat, the distal tip and threaded shaft of the anchor element extend through the opening at the distal end of the coupling element. Although shown and described by way of example as a screw, the anchor element could be any element capable of securing the coupling element to a bone segment, including but not limited to a screw, hook, staple, tack and/or suture.

The head further includes at least one pair of opposing slots extending at least partially through the head and in communication with the recess, dividing the head into two portions. When the head engages the seat, the seat will direct a compressive force on the head. Because the opposing slots divide head into head portions, the compressive force directed by the seat causes the head portions to be slightly biased toward one another. At the same time, the head portions are naturally resisting this compressive force and exerting its own radial force upon the seat. This interaction of forces creates a friction engagement between the head and the seat sufficient to allow the threaded shaft to overcome the effect of gravity yet remain easy to manipulate by a user. The result of this friction engagement is that the threaded shaft may be selectively moved by a user without requiring additional instrumentation to maintain the threaded shaft at a particular angle prior to insertion into bone. With a normal relationship between head and seat (i.e. head and seat having approximately equal diameters), the treaded portion will be acted upon by gravity and thus require additional instrumentation to maintain a particular angle.

By way of example only, the favored-angle bone screw assembly has a coupling element designed to pivot further in one direction than in others in order to achieve increased angulation over that available with a traditional polyaxial bone screw assembly. By positioning the coupling element such that the increased angulation is directed to place the coupling element more in line with the coupling elements of other vertebra, surgeons are able to minimize bending of the spine rods. The bone screw assembly of the present invention is further provided with visual elements which serve to identify the direction of the increased angulation.

According to one broad aspect of the present invention, the favored-angle bone screw assembly includes a coupling element, an anchoring element, a compression cap and a set screw. The coupling element is generally cylindrical in shape with a proximal end and a distal end. The distal end comprises a first generally planar surface and a second generally curved surface. The coupling element includes an axial bore extending axially therethrough from the proximal end to the distal end. At the distal end is an opening (formed at least partially within each of the first and second surfaces) with a diameter greater than that of the threaded portion of the anchoring element, but smaller than that of the head. The diameter of the axial bore is greater than that of the head of the anchoring element, so that the anchoring element may be guided through by its threaded portion going through the distal opening of the coupling element, and by the head going as far as the distal portion of the axial bore. The distal portion of the axial bore forms a seat for engaging the head, the seat being constructed as a partially spherically-shaped region corresponding to the size and shape of the underside of the head of the anchoring element. The coupling element further includes a pair of side extensions extending between the proximal end and distal end, and a U-shaped recess for receiving an orthopedic rod positioned between the side extensions. According to one embodiment of the present invention, the coupling element includes parallel planar faces on lateral sides. Because the distal end of the coupling element includes the second generally curved surface, one lateral side is shorter than the other lateral side. Within the axial bore toward the proximal end of the side extensions is a threaded region for engagement with a locking member constructed as a nut or, preferably, a set screw.

The anchoring element may be, by way of example only, a screw possessing a distal tip for insertion into bone, a head at the proximal end thereof, and a threaded portion extending between distal tip and head. The head is preferably sized and shaped to pass through the axial bore of the coupling element until the underside of the head engages the seat. The head has an underside that is preferably generally spherical in shape for engaging the seat. Although shown and described by way of example as a screw, the anchor element could be any element capable of securing the coupling element to a bone segment, including but not limited to a screw, hook, staple, tack and/or suture.

The compression cap, which is adapted to be positioned within the coupling element, has a generally cylindrical shape and includes a rod-receiving proximal surface. The distal surface is generally concave and adapted to engage a portion of the spherical head of the anchoring element. In one embodiment, the compression cap has a center bore extending from the proximal surface to the distal surface, that, when assembled, will help to fix the angular orientation of the coupling element in relation to the anchoring element by friction.

The set screw is also generally cylindrical in shape, with a proximal surface and a distal surface, and serves to fix the orthopedic rod within the U-shaped recesses. In one embodiment, the screw has a threaded section around the perimeter extending from the proximal surface to the distal surface. When assembled, the set screw serves to fix the orthopedic rod within the coupling member, which in turn pressures the compression cap and creates the friction between the compression cap and anchoring element necessary to fix the angular orientation of the coupling element in relation to the anchoring element.

The favored-angle bone screw assembly of the present invention provides a greater range of angulation between coupling element and anchoring element than can be obtained with a traditional polyaxial screw. Because the distal end of the coupling element of the favored-angle bone screw assembly includes the second generally curved surface, the increased angulation offered by the coupling element is biased in one direction. The largest maximum angulation is achieved with the anchoring element positioned towards the shorter lateral side of the coupling element. The smallest maximum angulation is achieved with the anchoring element positioned towards the longer lateral side. To facilitate the beneficial use of this biased directional angulation, the coupling element may be provided with visual indications to distinguish the shorter lateral side from the longer lateral side. In one embodiment of the present invention, signaling is accomplished by color coding the inner and/or outer surfaces of at least a portion of the half of the coupling element containing the shorter lateral side. Although described herein by way of example as color coding, signaling may also be accomplished by alternative visual indicia such as raised surfaces, notches or detents, partial coloration, laser-marked etchings, or other alterations or additions to the device that serve to demarcate the shorter lateral side.

By way of example only the laminar hook includes a housing portion and a bone-engaging portion. The housing portion includes a generally U-shaped recess dimensioned to receive at least a portion of the spinal rod. The housing portion also includes a threaded region dimensioned to receive a setscrew for securing the rod within the recess. The bone-engaging portion is generally provided as a hook-shaped member dimensioned to engage a portion of bone.

### BRIEF DESCRIPTION OF THE DRAWINGS

Many advantages of the present invention will be apparent to those skilled in the art with a reading of this specification in conjunction with the attached drawings, wherein like reference numerals are applied to like elements and wherein:

FIG. 1 is a perspective view of one example of a surgical fixation system according to first embodiment of the present invention;

FIG. 2 is a perspective view of one example of a surgical fixation system according to a second embodiment of the present invention;

FIG. 3 is a perspective view of an occipital plate engaged with a pair of spinal rods forming part of the surgical fixation system of FIG. 1;

FIG. 4 is a perspective view of the occipital plate of FIG. 3;

FIG. 5 is a front plan view of the occipital plate and spinal rods of FIG. 3;

FIG. 6 is a front plan view of the occipital plate of FIG. 4;

FIG. 7 is a side view of the occipital plate of FIG. 4;

FIG. 8 is a side partial cross-sectional view of the occipital plate of FIG. 4;

FIG. 9 is a top view of the occipital plate of FIG. 4;

FIG. 10 is a bottom view of the occipital plate of FIG. 4;

FIG. 11 is a perspective view of an eyelet connector attached to a spinal rod forming part of the surgical fixation system of FIG. 2;

FIG. 12 is a perspective view of the eyelet connector of FIG. 11;

FIG. 13 is a top plan view of the eyelet connector and spinal rod of FIG. 11;

FIG. 14 is a top plan view of the eyelet connector of FIG. 11;

FIG. 15 is a front view of the eyelet connector of FIG. 11;

FIG. 16 is a side view of the eyelet connector of FIG. 11;

FIG. 17 is a perspective view of a crosslink connector attached to a pair of spinal rods forming part of the surgical fixation system of FIG. 1;

FIG. 18 is a front plan view of the crosslink connector of FIG. 17;

FIG. 19 is a top plan view of the crosslink connector of FIG. 17;

FIG. 20 is a side cross-sectional view of the crosslink connector of FIG. 17;

FIG. 21 is a perspective view of a friction-fit pedicle screw assembly forming part of the surgical fixation system of FIG. 1;

FIG. 22 is a side view of a bone screw forming part of the friction-fit pedicle screw assembly of FIG. 21;

FIG. 23 is a perspective view of the bone screw of FIG. 22;

FIG. 24 is a top view of the bone screw of FIG. 22;

FIG. 25 is a side view of a housing forming part of the friction-fit pedicle screw assembly of FIG. 21;

FIG. 26 is a side cross-sectional view of the housing of FIG. 25;

FIG. 27 is a side cross-sectional view of the friction-fit pedicle screw assembly of FIG. 21;

FIG. 28 is a partial cross-sectional view the friction-fit pedicle screw assembly of FIG. 21;

FIG. 29 is an exploded perspective view of one example of a favored-angle bone screw assembly forming part of the surgical fixation system of FIG. 1;

FIG. 30 is a perspective view of the favored-angle bone screw assembly of FIG. 29, fully assembled and coupled to a spinal rod;

FIG. 31 is a side view of a prior art polyaxial bone screw assembly;

FIGS. 32 and 33 are side views of the bone screw assembly of FIG. 30, illustrating the asymmetrical angulation that is achieved by the present invention;

FIG. 34 is a perspective view of a coupling element forming part of the favored-angle bone screw assembly of FIG. 29;

FIGS. 35-37 are top, front, and side views, respectively, of the coupling element of FIG. 34; and

FIGS. 38-39 are perspective and side views, respectively, of a laminar hook forming part of the surgical fixation system of FIG. 1.

### DESCRIPTION OF THE PREFERRED EMBODIMENT

Illustrative embodiments of the invention are described below. In the interest of clarity, not all features of an actual implementation are described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure. The surgical fixation system disclosed herein boasts a variety of inventive features and components that warrant patent protection, both individually and in combination.

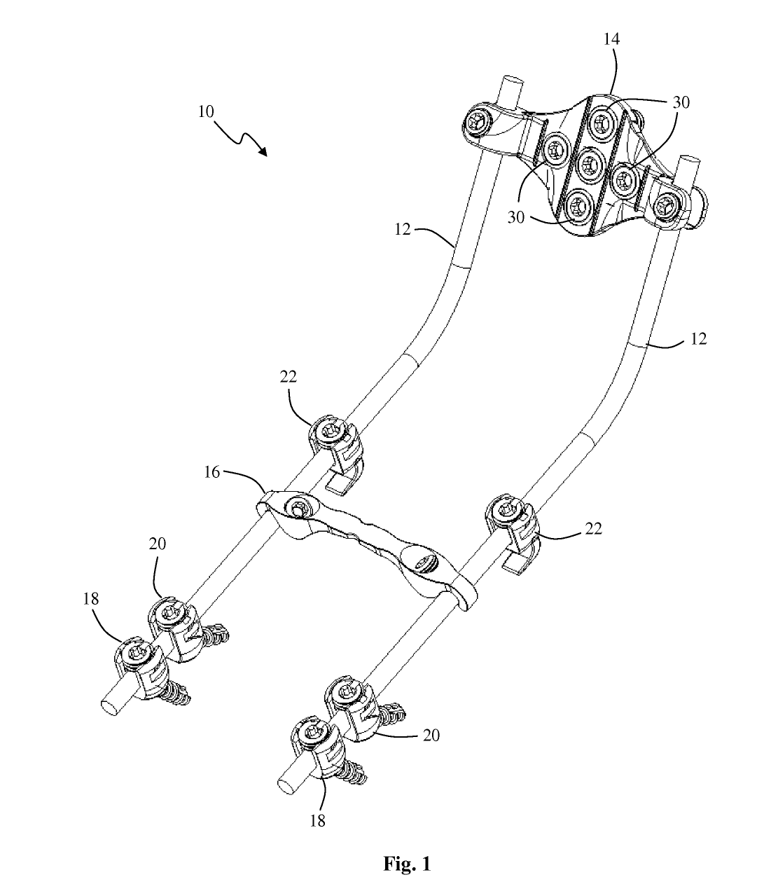

FIG. 1 illustrates one example of a surgical fixation system 10 according a first embodiment of the present invention. The surgical fixation system 10 includes a pair of spinal rods 12, an occipital plate 14, a crosslink connector 16, and a plurality of anchor elements, including but not limited to (and shown by way of example only) friction-fit pedicle screws 18, favored-angle pedicle screws 20, and hooks 22. Any or all of these elements may be made of a biologically inert material, preferably any metal customarily used for surgical devices, such as for example titanium or stainless steel. The surgical fixation system 10 of the present invention is described herein for application to the posterior region of the human spine, for attachment to cervical and/or thoracic vertebrae, as well as the occiput portion of the skull. However, it should be noted that a surgical fixation system 10 of the type described here may find application to other parts of the body.

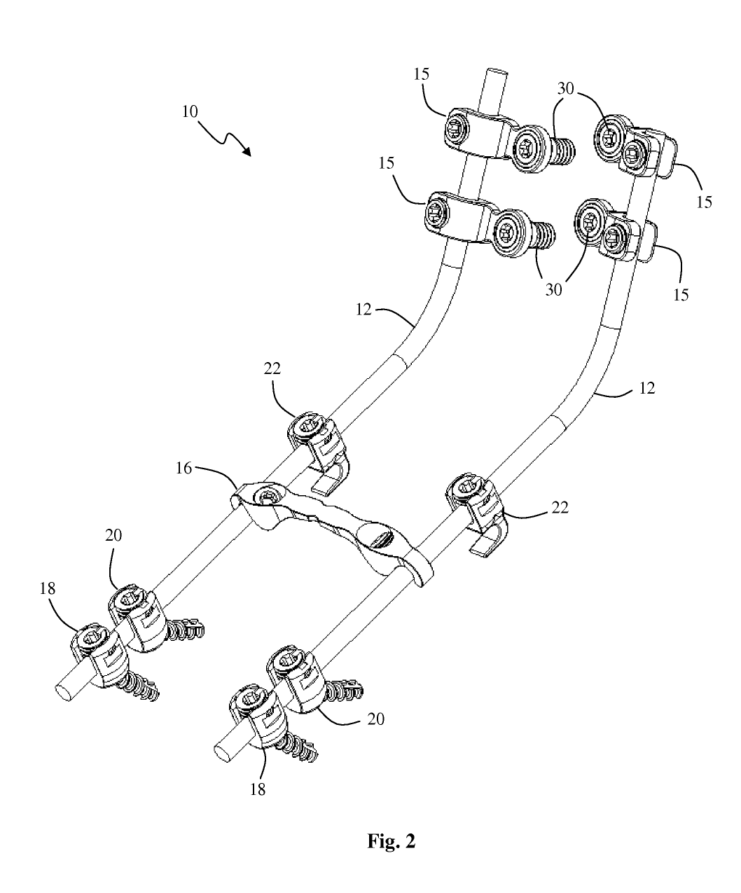

FIG. 2 illustrates one example of a surgical fixation system 11 according to a second embodiment of the present invention. For the sake of simplicity, features and elements identical to both surgical fixation systems 10, 11 have been assigned identical callout numbers. Thus, surgical fixation system 11 includes a pair of spinal rods 12, plurality of occipital anchors 15, a crosslink connector 16, and a plurality of spinal anchor elements, including but not limited to (and shown by way of example only) friction-fit pedicle screws 18, favored-angle pedicle screws 20, and hooks 22. Any or all of these elements may be made of a biologically inert material, preferably any metal customarily used for surgical devices, such as for example titanium or stainless steel. The surgical fixation system 11 of the present invention is described herein for application to the posterior region of the human spine, for attachment to cervical and/or thoracic vertebrae, as well as the occiput portion of the skull. However, it should be noted that a surgical fixation system 10 of the type described here may find application to other parts of the body.

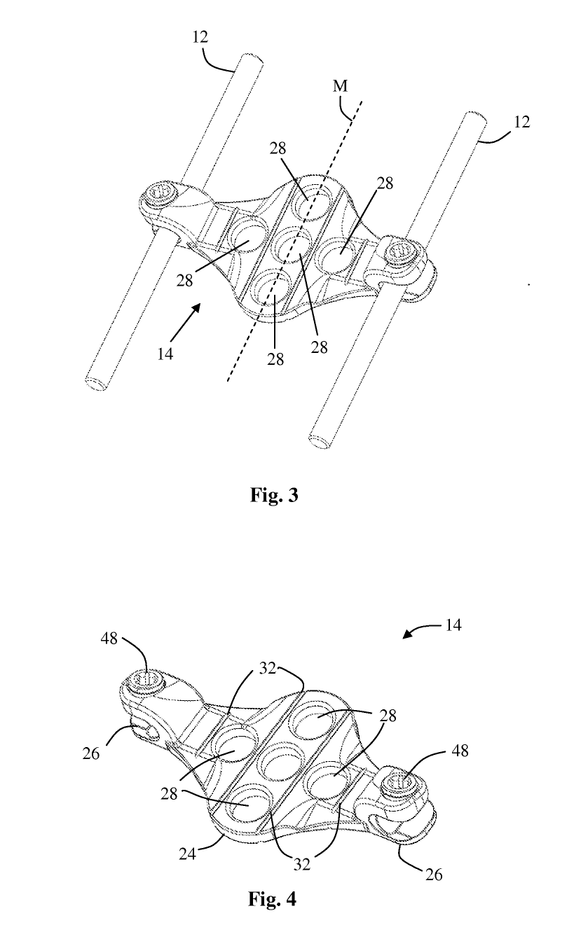

Referring to FIGS. 3-10, the occipital plate 14 of the surgical fixation system 10 comprises a generally flat body portion 24 flanked by a pair of side-loading clamp elements 26, each dimensioned to receive one of the spinal rods 12. The body portion 24 may have any shape suitable to facilitate secure attachment of the occipital plate 14 to the occiput region of a patient's skull, including but not limited to the rounded diamond shape shown by way of example only in FIGS. 3-10. The body portion 24 includes a plurality of apertures 28, each dimensioned to receive an anchor element such as an occipital screw 30 (shown in FIG. 1). By way of example only, the occipital plate 14 as shown is provided with five apertures 28, with three of the apertures 28 aligned along the longitudinal midline M and an additional aperture 28 laterally offset on either side of the longitudinal midline M, as shown in FIG. 3. The body portion 24 further includes a plurality of longitudinal grooves 32 cut into the top and bottom surfaces 34, 36 extending in a direction generally parallel to that of the spinal rods 12. The longitudinal grooves 30 function to provide regions of flexibility such that the occipital plate 14 of the present invention is selectively customizable to fit the contours of the particular patient. Specifically, the grooves 30 provide regions along which portions of the occipital plate 14 are relatively easily bendable, without endangering the structural integrity of the apertures 28.

As best seen in FIGS. 9 and 10, the clamp elements 26 extend laterally (and generally opposite one another) from the body portion 24. The clamp elements 26 on either side of occipital plate 14 are essentially mirror images of one another, and thus for simplicity only one clamp element 26 will be described in detail. Clamp element 26 comprises a first clamp portion 38 and a second clamp portion 40. The first clamp portion 38 is a generally flat extension of the bottom surface 36, while the second clamp portion 40 is a curved element protruding generally perpendicularly out of the top surface 34 such that the first and second clamp portions 38, 40 together form a generally U-shaped channel 42 therebetween. Channel 42 is dimensioned to receive at least a portion of spinal rod 12, and first clamp portion 38 includes a detent 44 within channel 42 in order to allow a “snap-fit” engagement between the clamp element 26 and spinal rod 12. In order to further secure the rod 12 within the clamp element 26, the second clamp portion 40 includes an aperture 46 dimensioned to receive a setscrew 48, which functions as a locking element to secure the spinal rod 12 in place. In order to achieve this locking interaction, the setscrew 48 threadedly engages the aperture 46 such that the setscrew 48 may be advanced toward the spinal rod 12 until an angled surface 50 located at the distal tip of the setscrew 48 contacts the rod. In practice, the setscrew 48 may be advanced to an extent such that the angled surface 50 causes a slight deformation in the spinal rod 12, thereby preventing the rod 12 from being expelled from the channel 42 and effectively locking the spinal rod 12 to the occipital plate 14.

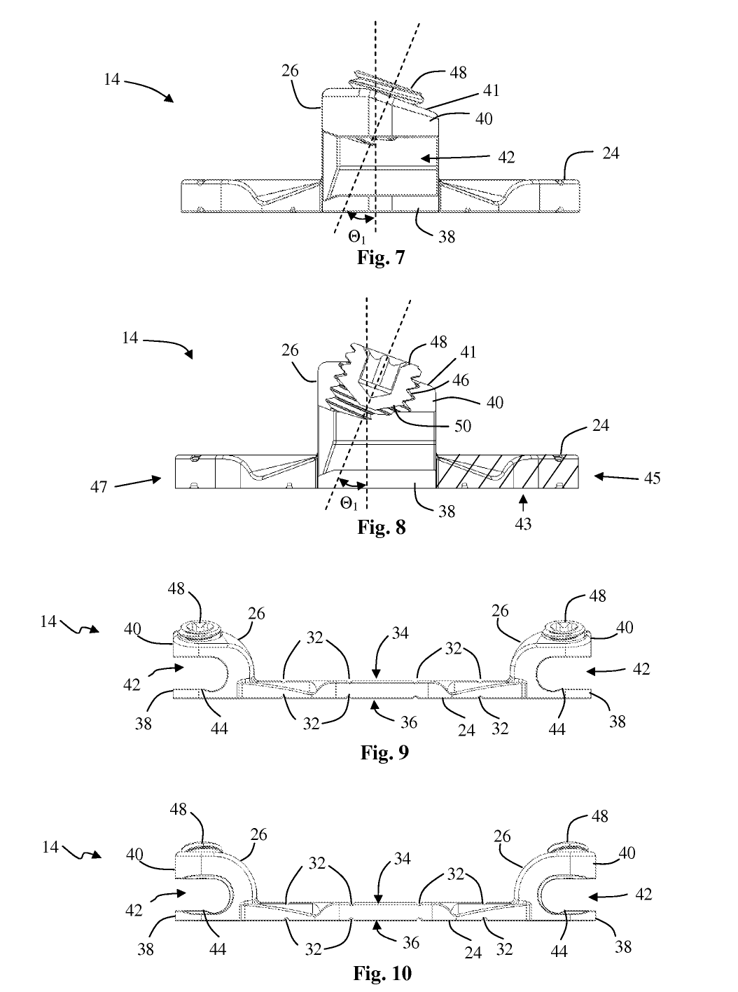

With specific reference to FIGS. 7 and 8, apertures 46 may be provided at an angle Θ₁ offset from the perpendicular extension of second clamp portion 40. In the example shown, the angle Θ₁ is approximately 20°, however it is contemplated that angle Θ₁ could include any angle within the range of 10° to 85° cranially offset from an axis perpendicular to the first clamp portion 38. For the purposes of this disclosure, “cranial” means toward the top of the head, and “caudal” means toward the feet. To further facilitate biased directional the angulation of apertures 46, the second clamp portion 40 has an upper surface 41 that is angled in a cranial direction. Providing apertures 46 at an angle Θ₁ provides occipital plate 14 with an improvement in that the screw insertion becomes significantly less troublesome for surgeons to perform, due to the natural curvature of a patient's spine. Specifically, the angled offset provides full visibility (e.g. direct line of sight for the surgeon) and increased access to the setscrew 48 from the surgeon's point of view when locking the spinal rod 12 to the occipital plate 14.

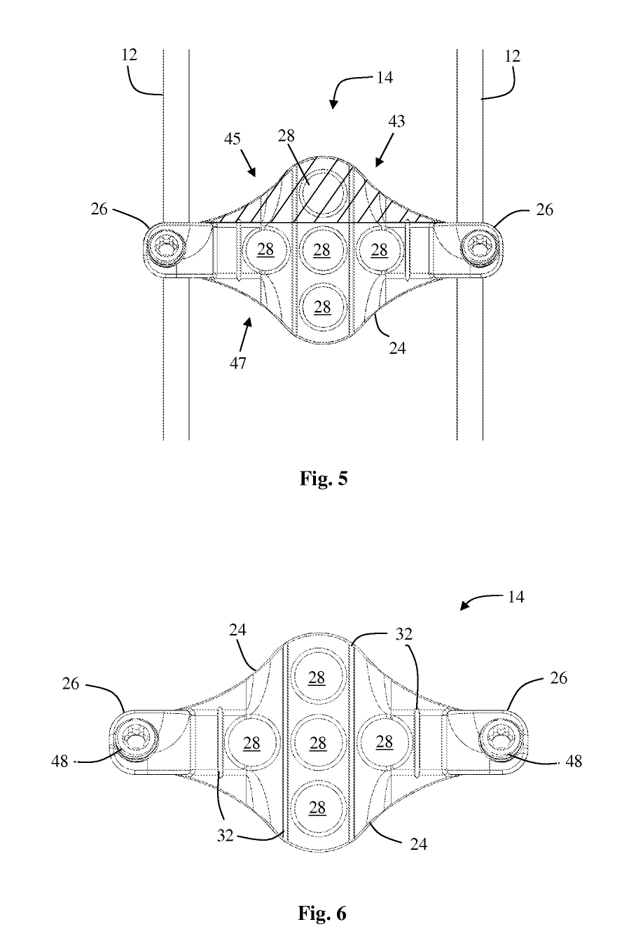

The benefits of the biased directional angulation described above require proper orientation of the occipital plate 14 when implanted on a patient's skull. Specifically, proper orientation of the occipital plate 14 is achieved when the biased directional angulation is provided in a cranial direction. This will ensure the bias is angled toward the surgeon. In order to facilitate the beneficial use of this biased directional angulation Θ₁, the occipital plate 14 may be provided with a visual indication 43 to distinguish the cranial side 45 of the body portion 24 from the caudal side 47 (and thus the indicate the direction of the biased angulation Θ₁), as shown in the example provided in FIGS. 5 and 8. By way of example only, such visual indication may be accomplished by color coding the surfaces of at least a portion of the cranial side 45 of the occipital plate 14. The color coding is indicated in FIGS. 5 and 8 by diagonal lines on the surfaces of the cranial side 45. Although shown and described herein by way of example as color coding, other suitable visual indications 43 may be used, for example such as raised surfaces, notches or detents, partial coloration, laser-marked etchings, or other alterations or additions to the device that serve to demarcate the cranial side 45 of the occipital plate 14 and thereby the direction of the biased angulation Θ₁. Thus, the use of the visual indication 43 ensures proper orientation of the occipital plate 14 on the patient's skull.

The occipital plate 14 may be provided in any size suitable for any particular patient. By way of example only, the occipital plate 14 has a length (transverse to the longitudinal midline) measured from the center of each spinal rod 12 (when inserted) ranging between 35 mm and 45 mm, inclusive. However length dimensions provided outside the exemplary range are possible without departing from the scope of the present invention. The occipital screws 30 may be provided having any diameter and length dimension suitable for implantation into a patient's skull. By way of example only, the occipital screws 30 have a diameter ranging between 4.5 mm and 5.0 mm, inclusive, and a length dimension ranging between 6 mm and 14 mm, inclusive.

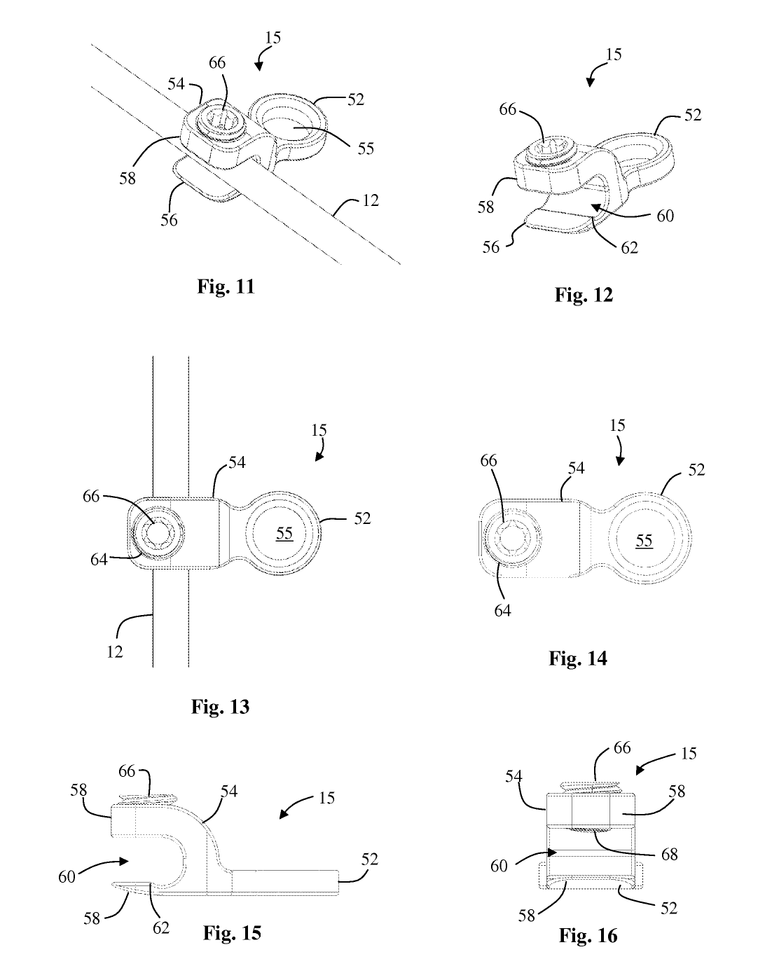

FIGS. 11-16 illustrate alternative occipital anchors 15 forming part of the surgical fixation system 11 of the present invention. By way of example only, occipital anchors 15 include an occipital fixation portion 52 connected to a clamp element 54 dimensioned to receive one of the spinal rods 12. The occipital fixation portion 52 includes an aperture 55 dimensioned to receive an occipital screw 30 (shown in FIG. 2) to facilitate attachment of the occipital anchor 15 to an occiput forming part of a patient's skull. Clamp element 54 comprises a first clamp portion 56 and a second clamp portion 58. The first clamp portion 56 is a generally flat extension of the occipital fixation portion 52, while the second clamp portion 58 is a curved element protruding generally perpendicularly out of the occipital fixation portion 52 such that the first and second clamp portions 56, 58 together form a generally U-shaped channel 60 therebetween. Channel 60 is dimensioned to receive at least a portion of spinal rod 12, and first clamp portion 56 includes a detent 62 within channel 60 in order to allow a “snap-fit” engagement between the clamp element 54 and spinal rod 12. In order to further secure the rod 12 within the clamp element 54, the second clamp portion 58 includes an aperture 64 dimensioned to receive a setscrew 66, which functions as a locking element to secure the spinal rod 12 in place. In order to achieve this locking interaction, the setscrew 66 threadedly engages the aperture 64 such that the setscrew 66 may be advanced toward the spinal rod 12 until the distal tip 68 of the setscrew 66 contacts the rod 12. In practice, the setscrew 66 may be advanced to an extent such that the distal tip 68 causes a slight deformation in the spinal rod 12, thereby preventing the rod 12 from being expelled from the channel 60 and effectively locking the spinal rod 12 to the occipital anchor 15.

Although shown as a generally direct approach, it should be understood that, like with the occipital plate 14 described above, apertures 64 may be provided at an angle offset from the perpendicular extension of second clamp portion 58. It is contemplated that this angle could include any angle within the range of 0° to 85° offset from the perpendicular extension of second clamp portion 58. Providing apertures 64 at such an angle provides occipital anchors 15 with an improvement in that the screw insertion becomes significantly less troublesome for surgeons to perform, due to the natural curvature of a patient's spine.

Occipital anchors 15 provide an alternative type of occipital fixation than that of the occipital plate 14 described above. One advantage of the occipital anchors 15 is the increased flexibility of occipital screw 30 placement due to the independent placement of the occipital anchors 15. Moreover, this flexibility is enhanced by the freedom of movement of the occipital anchors 15 relative to the spinal rod 12. Once engaged to the rod 12 (and before insertion of the occipital screw 30), the occipital anchor 15 exhibits three degrees of freedom to facilitate optimal placement of the occipital screws 30. First, the occipital anchors 15 may translate longitudinally along the spinal rods 30 to allow the surgeon to locate the optimal location on the patient's skull for the placement of occipital screws 30. Second, the occipital anchors 15 may pivot dorsally about the spinal rod 12. Lastly, the occipital anchors 15 may pivot ventrally about the spinal rod 12 to facilitate optimal fixation of the spinal rod 12 to the patient's occiput.

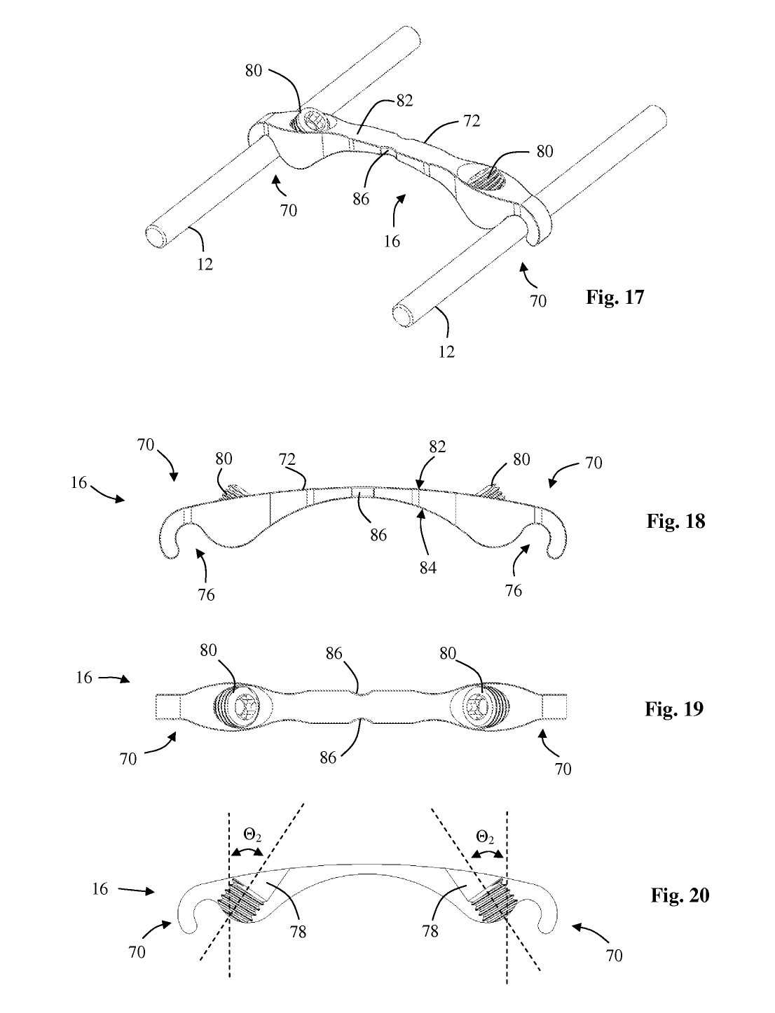

FIGS. 17-20 illustrate one example of a crosslink connector 16 forming part of the surgical fixation systems 10, 11 of the present invention. Crosslink connector 16 is provided as a unitary member having a pair of opposing clamp portions 70 separated by an elongated central portion 72. Each clamp portion 70 includes curved extension 74 forming a channel 76 dimensioned to receive at least a portion of the spinal rod 12 therein. Clamp portion 70 further includes aperture 78 dimensioned to threadedly receive a setscrew 80 for locking the spinal rod 12 within channel 76. Aperture 78 may be provided such that its longitudinal axis is medially offset at an angle Θ₂ relative to an axis extending perpendicularly from the longitudinal axis of the spinal rod 12, as illustrated in FIG. 20. By way of example only, the angle Θ₂ may be approximately 45°. This disposition of the apertures 78 is advantageous in that it allows for a more direct approach for inserting the setscrews 80.

Crosslink connector 16 is provided as a generally arched member including a first concave surface 82 having a width and a first degree of curvature, and a second concave surface 84 having a width and a second degree of curvature different from the first degree of curvature. The generally arched nature of the crosslink connector allows it to traverse the cervical spine without interfering with spinal structures. Crosslink connector 16 further includes at least one pair of opposing indentations 86 along the elongated central portion 72. As shown, the crosslink connector 16 includes one pair of opposing indentations 86 at the approximate midpoint of the central portion 72. Opposing indentations 86 provide for customizable bending of the crosslink connector 16 in a number of directions to fit the particular needs of a user. The crosslink connector 16 may be provided in any length suitable for extending between spinal rods 12. By way of example only, crosslink connector 16 may have any length within the range of 26 mm and 50 mm, inclusive.

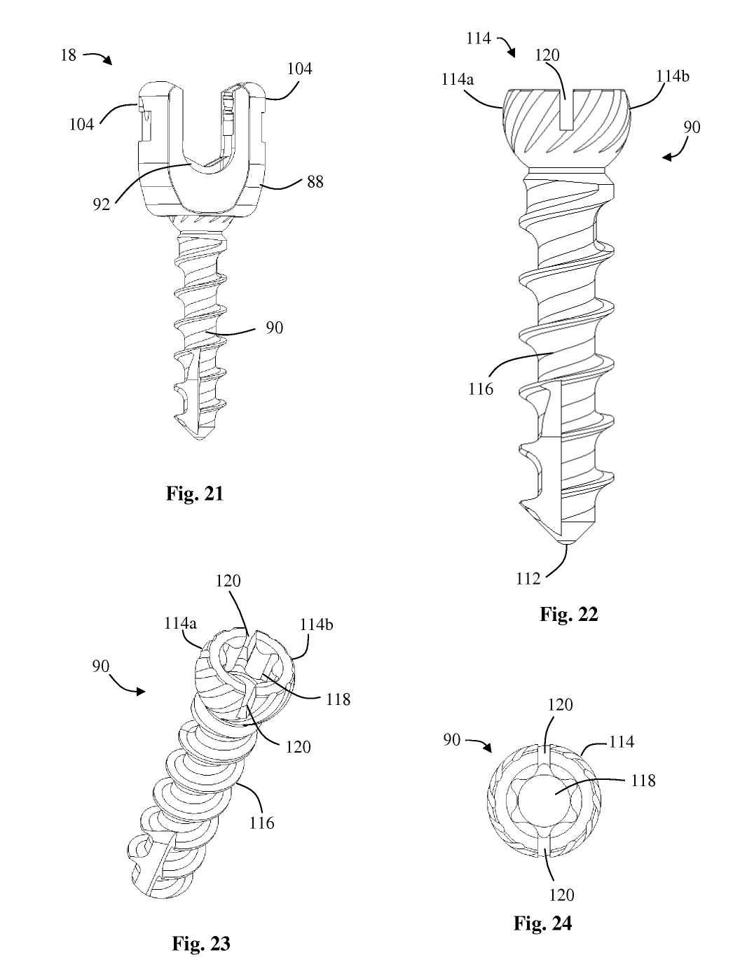

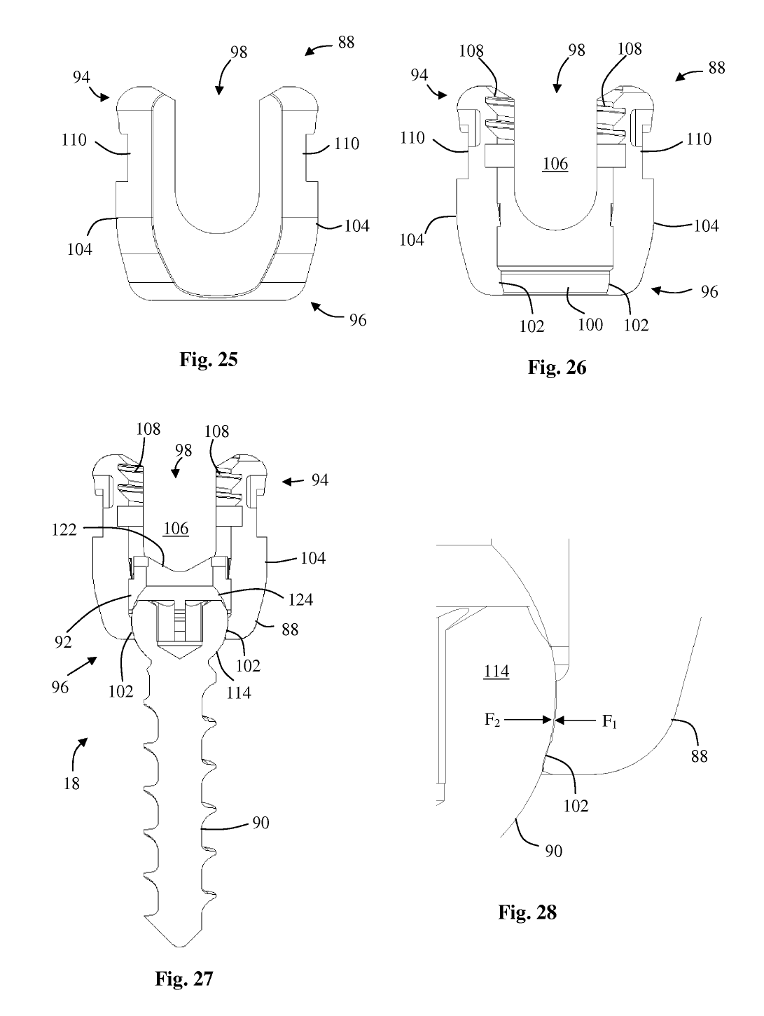

FIGS. 21-27 illustrate one example of a friction-fit polyaxial pedicle screw assembly 18 according one embodiment of the present invention. The friction-fit polyaxial pedicle screw assembly 18 includes a coupling element 88, an anchor element 90, a compression cap 92 and a set screw (not shown).

The coupling element 88, shown in detail in FIGS. 22 and 26, is generally cylindrical in shape with a proximal end 94 and a distal end 96. The coupling element 88 includes a passage 98 extending axially therethrough from the proximal end 94 to the distal end 96. At the distal end 96 is an opening 100 dimensioned to permit passage of the threaded portion 116 of the anchor element 90, but not the head 114 of the anchor element 90. The distal portion of the passage 98 forms a seat 102 for engaging the head 114, the seat 102 being constructed so as to receive a partially spherically shaped region corresponding to the size and shape of the head 114 of the anchor element 90. The seat 102 is also constructed as having a diameter slightly smaller than that of the corresponding portion of the head 114 of the anchor element 90. The coupling element 88 further includes a pair of side extensions 104 extending between the proximal end 94 and distal end 96, and a U-shaped recess 106 for receiving a least a portion of the spinal rod 12 positioned between the side extensions 104. Within the passage 98 toward the proximal end 94 is a threaded region 108 for threaded engagement with a locking member constructed as a nut or, preferably, a set screw (not shown). The coupling element 88 may include one or more notches or detents 110 on the side extensions 104 for engaging an insertion device (not pictured).

Referring to FIGS. 23-25, the anchor element 90 is shown by way of example only as a screw including a distal tip 112 for insertion into bone, a head 114 at the proximal end thereof, and threaded shaft 116 extending between distal tip 112 and head 114. The head 114 may include a recess 118 adapted to cooperate with a driver used to sink the anchor element 90 into bone. By way of example only, the recess 118 is shown as a hex-head recess for receiving a hex-head driver. The head 114 is preferably sized and shaped to pass through the passage 98 of the coupling element 88 until the head 114 engages the seat 102. The head 118 is generally spherical in shape and dimensioned to engage the seat 102. When the head 114 engages the seat 102, the distal tip 112 and threaded shaft 116 of the anchor element 90 extend through the opening 100 at the distal end 96 of the coupling element 88. Although shown and described by way of example as a screw, the anchor element 90 could be any element capable of securing the coupling element 88 to a bone segment, including but not limited to a screw, hook, staple, tack and/or suture.

The head 114 further includes at least one pair of opposing slots 120 extending at least partially through the head and in communication with the recess 118, dividing the head 114 into two portions 114a, 114b, as shown in FIGS. 23 and 24. As previously described, the seat 102 is constructed as having a diameter slightly smaller than that of the corresponding portion of the head 114 of the anchor element 90. Thus, when the head engages the seat 102, the seat 102 will direct a compressive force F₁ (FIG. 28) on the head 114. Because the opposing slots 120 divide head 114 into head portions 114a, 114b, the compressive force F₁ directed by the seat 102 causes head portions 114a, 114b to be slightly biased toward one another. At the same time, head portions 114a, 114b are naturally resisting this compressive force F₁ and exerting its own radial force F₂ upon the seat 102. This interaction of forces creates a friction engagement between the head 114 and the seat 102 sufficient to allow the threaded shaft 116 to overcome the effect of gravity yet remain easy to manipulate by a user. The result of this friction engagement is that the threaded shaft 116 may be selectively moved by a user without requiring additional instrumentation to maintain the threaded shaft 116 at a particular angle prior to insertion into bone. With a normal relationship between head and seat (i.e. head and seat having approximately equal diameters), the treaded portion will be acted upon by gravity and thus require additional instrumentation to maintain a particular angle.

The compression cap 92, which is adapted to be positioned within the coupling element 88, has a generally cylindrical shape and includes a rod-receiving proximal surface 122. The distal surface 124 is generally concave and adapted to engage a portion of the head 114 of the anchor element 90. Upon insertion of the spinal rod 12, the compression cap 92 functions to help to fix the angular orientation of the coupling element 88 in relation to the anchor element 90 by friction.

The set screw (not shown) is dimensioned to engage threaded region 108 and serves to fix the spinal rod 12 within the coupling member 88, which in turn pressures the compression cap 92 and creates the friction between the compression cap 92 and anchor element 90 necessary to fix the angular orientation of the coupling element 88 in relation to the anchor element 90.

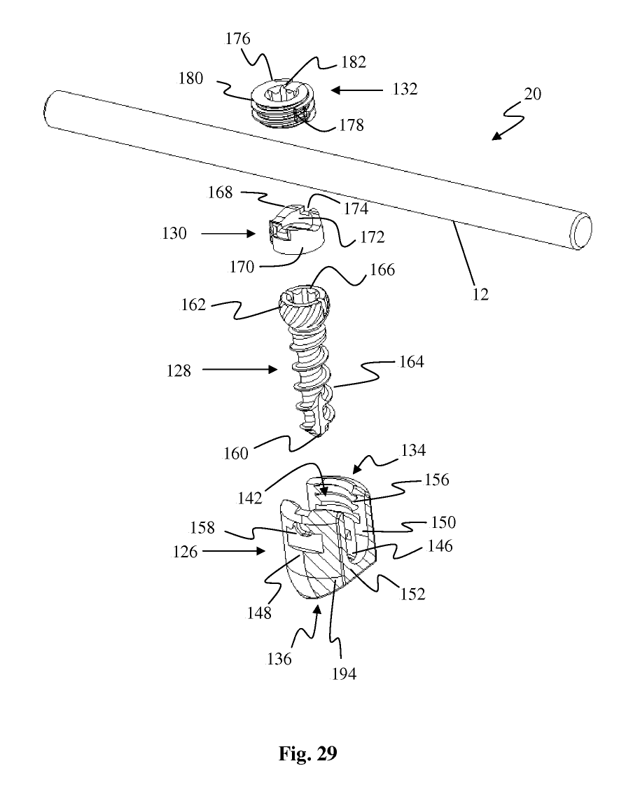

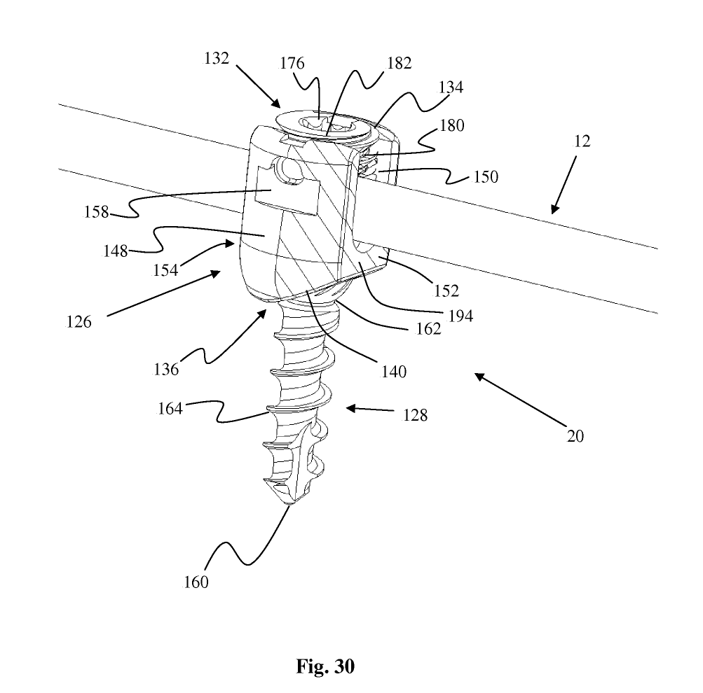

FIGS. 29-37 illustrate one example of a favored-angle pedicle screw 20 forming part of surgical fixation systems 10, 11 according to one embodiment of the present invention. Referring to FIGS. 29 and 30, the favored-angle pedicle screw 20 includes a coupling element 126, an anchoring element 128, a compression cap 130 and a set screw 132. Any or all of these elements may be made of a biologically inert material, preferably any metal customarily used for surgical devices, such as for example titanium or stainless steel.

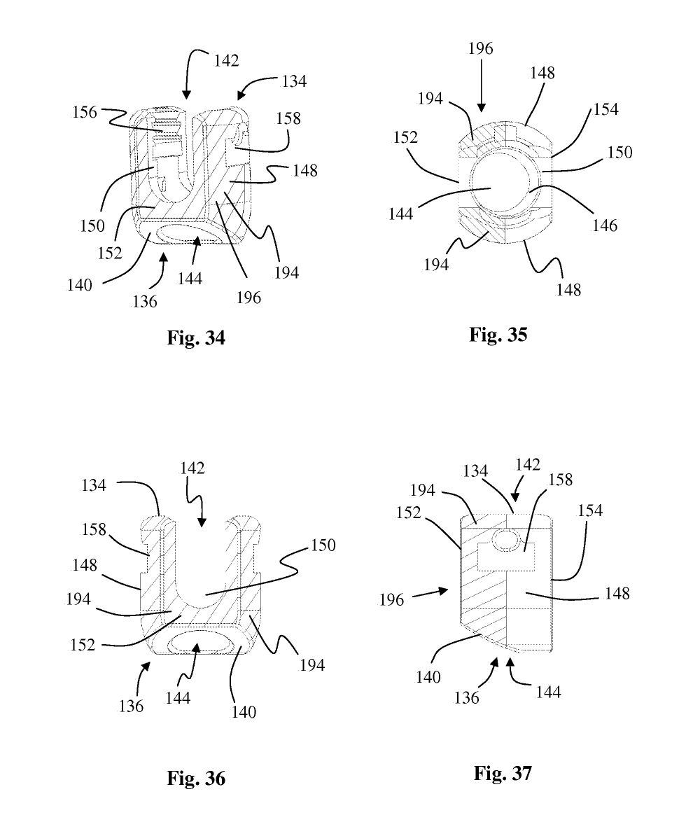

The coupling element 126, shown in detail in FIGS. 34-37, is generally cylindrical in shape with a proximal end 134 and a distal end 136. The distal end 136 comprises a first generally planar surface 138 and a second generally curved surface 140. The coupling element 126 includes an axial bore 142 extending axially therethrough from the proximal end 134 to the distal end 136. At the distal end 136 is an opening 144 (formed at least partially within each of the first and second surfaces 138, 140) with a diameter greater than that of the threaded portion 164 of the anchoring element 14, but smaller than that of the head 162. The diameter of the axial bore 142 is greater than that of the head 162, so that the anchoring element 128 may be guided through by its threaded portion 164 going through the opening 144, and by the head 162 going as far as the distal portion of the axial bore 142. The distal portion of the axial bore 142 forms a seat 146 for engaging the head 162, the seat 146 being constructed so as to receive a partially spherically shaped region corresponding to the size and shape of the underside of the head 162 of the anchoring element 128. The coupling element 126 further includes a pair of side extensions 148 extending between the proximal end 134 and distal end 136, and a U-shaped recess 150 for receiving an orthopedic rod 12 positioned between the side extensions 148. In the example shown in FIG. 29, the coupling element 126 includes parallel planar faces on first and second lateral sides 152, 154. Because the distal end 136 of the coupling element 126 includes the second generally curved surface 140, first lateral side 152 is shorter than second lateral side 154. Within the axial bore 142 toward the proximal end 134 of the side extensions 148 is a threaded region 156 for engagement with a locking member constructed as a nut or, preferably, a set screw 132. The coupling element 126 may include one or more notches or detents 158 on the side extensions 148 for engaging an insertion device (not pictured).

Referring again to FIG. 29, the anchoring element 128 is shown by way of example only as a screw including a distal tip 160 for insertion into bone, a head 162 at the proximal end thereof, and threaded portion 164 extending at between distal tip 160 and head 162. The head 162 may include one or more depressions or grooves 166 adapted to cooperate with a driver used to sink the anchoring element 128 into bone. The head 162 is preferably sized and shaped to pass through the axial bore 142 of the coupling element 126 until the underside of the head 162 engages the seat 146. The head 162 has an underside that is preferably generally spherical in shape for engaging the seat 146. When the underside of the head 162 engages the seat 146, the distal tip 160 and threaded portion 164 of the anchoring element 128 extend through the opening 144 at the distal end 136 of the coupling element 126. Although shown and described as having a threaded portion 164 extending fully between the distal tip 160 and head 162, other configurations are possible without departing from the scope of the present invention. For example, an anchor element 128 could be provided having a shaft extending between the distal tip and head, with the shaft being partially threaded and partially unthreaded. The ratio of threaded portion to unthreaded portion can vary depending upon the particular needs of the surgeon, however by way of example only the ratio of threaded to unthreaded portion may be 1:1 (in other words the anchoring element 128 may be provided having an equal amount of threaded and unthreaded portions). Although shown and described by way of example as a screw, the anchor element 128 could be any element capable of securing the coupling element 126 to a bone segment, including but not limited to a screw, hook, staple, tack and/or suture.

The compression cap 130, which is adapted to be positioned within the coupling element 126, has a generally cylindrical shape and includes a rod-receiving proximal surface 168. The distal surface 170 is generally concave and adapted to engage a portion of the spherical head 162 of the anchoring element 128. In one embodiment, the compression cap 130 has a center bore 172 extending from the proximal surface 168 to the distal surface 170, that, when assembled, will help to fix the angular orientation of the coupling element 126 in relation to the anchoring element 128 by friction. The compression cap 130 may also include notches or detents 174 on the proximal surface 168 for engaging an insertion device (not pictured).

The set screw 132 is also generally cylindrical in shape, with a proximal surface 176 and a distal surface 178, and serves to fix the orthopedic rod 12 within the U-shaped recesses 150. In one embodiment, the screw has a threaded section 180 around the perimeter extending from the proximal surface 176 to the distal surface 178. It is possible for the set screw 132 to engage the rod 12 directly or via a pressure member (not pictured). The set screw 132 generally has one or more depressions or grooves 182 adapted to cooperate with a driver to cause the set screw 132 to engage the rod 12. When assembled, the set screw 132 serves to fix the orthopedic rod 12 within the coupling member 126, which in turn pressures the compression cap 130 and creates friction between the compression cap 130 and anchoring element 128 necessary to fix the angular orientation of the coupling element 126 in relation to the anchoring element 128. The set screw 132 may also include a feature for locking its position once engaged within the coupling member 126, such as a pin or snap ring (not pictured).

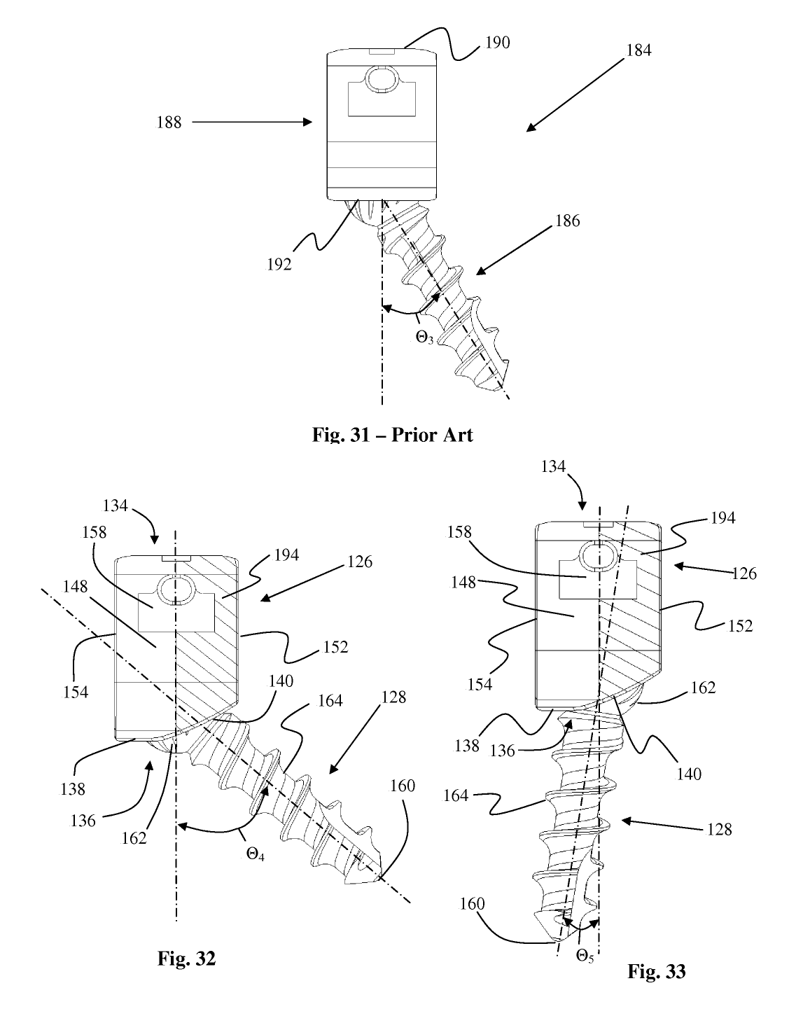

FIG. 31 illustrates an example of a regular polyaxial bone screw assembly 184 of the type commonly used in the art. Polyaxial bone screw assembly 184 includes an anchoring element 186, a coupling element 188, a compression cap (not pictured), and a set screw (not pictured). The proximal end 190 and distal end 192 of the coupling element 188 form generally parallel planes, thereby creating a maximum angles Θ₃ by which the anchoring element 186 may be offset from the axis of the coupling element 188. Generally, such polyaxial screw assemblies 184 provide no more than 40° of angulation Θ₃ in any direction, and the angulation is generally symmetrical in nature. Thus, rotating the tip of the anchoring element 186 in a maximum circumferential path creates a zone of angulation that is symmetrical in shape (e.g. generally conical).

As shown in FIGS. 32 and 33, the favored-angle pedicle screw 20 of the present invention provides a greater range of angulation Θ₄ between coupling element 126 and anchoring element 128 than can be obtained with a traditional polyaxial screw 184. Because the distal end 136 of the coupling element 126 of the favored-angle pedicle screw 20 includes the second generally curved surface 140, the increased angulation Θ₄ offered by the coupling element 126 is biased in one direction. In other words, the angulation is generally asymmetrical such that rotating the tip 160 of the anchoring element 128 in a maximum circumferential path creates an asymmetrical zone of angulation that is biased in one direction. The largest maximum angulation Θ₄ is achieved with the anchoring element 128 positioned towards the shorter first lateral side 152 of the coupling element 126, as shown in FIG. 32. The smallest maximum angulation Θ₅ is achieved with the anchoring element 128 positioned towards the longer second lateral side 154 as shown in FIG. 33. To facilitate the beneficial use of this biased directional angulation Θ₄, the coupling element 126 may be provided with a visual indication 194 to distinguish the shorter lateral side 152 from the longer lateral side 154 (and thus the direction of the biased angulation Θ₅), as shown in the example provided in FIGS. 34-37. By way of example only, such visual indication may be accomplished by color coding the inner and/or outer surfaces of at least a portion of the first half 196 of the coupling element 126 (i.e. the half 196 containing the shorter first lateral side 152). The color coding is indicated in FIGS. 34-37 by diagonal lines on the surfaces of the first half 196. Although shown and described herein by way of example as color coding, other suitable visual indications 194 may be used, for example such as raised surfaces, notches or detents, partial coloration, laser-marked etchings, or other alterations or additions to the device that serve to demarcate the shorter first lateral side 152.

The maximum biased directional angulation Θ₄ achieved by bone screw assembly of the present invention, as shown in FIG. 32 is approximately 55°, and the smallest maximum angulation Θ₅, show in FIG. 33, is approximately 10°. These angles are shown and described by way of example only, and in practice, favored-angle pedicle screw 20 may be provided with any maximum biased directional angulation Θ₄ that would be useful and/or required, for example any angle within a range of 40-65°. Likewise, favored-angle pedicle screw 20 may be provided with any suitable smallest maximum angulation Θ₅, for example any angle within a range of 5-30°. The visual signaling elements of the present invention can serve to distinguish the maximum biased directional angulation Θ₄ of a spinal fixation assembly 10, 11 achieving angulation of any degree, where two or more different degrees of biased directional angulation are provided.

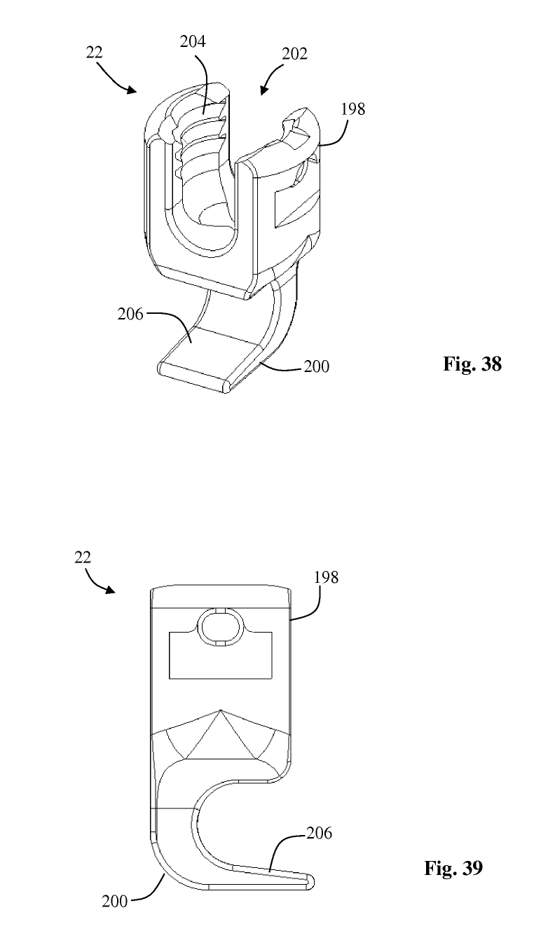

FIGS. 38-39 illustrate one example of a laminar hook 22 forming part of surgical fixation systems 10, 11 according to one embodiment of the present invention. Hook 22 includes a housing portion 198 and a bone-engaging portion 200. Housing portion 198 includes a generally U-shaped recess 202 dimensioned to receive at least a portion of the spinal rod 12. The housing portion 198 also includes a threaded region 204 dimensioned to receive a setscrew (not shown) for securing the rod 12 within the recess 202. The bone-engaging portion 200 is generally provided as a hook-shaped member 206 dimensioned to engage a portion of bone.

In use, either the occipital plate 14 or a plurality of occipital anchors 15 are attached to the occiput region of a patient's skull using a plurality of occipital screws 30. If the occipital plate 14 is used, then the visual indicator 43 is placed facing cranially to ensure proper positioning of the occipital plate 14. Spinal rods 12 are then secured to the occipital plate 14 or occipital anchors 15 by the methods described above. The rods 12 are then extended along the posterior aspects of the patient's cervical and potentially thoracic spine on either side of the spinous processes for a desired distance. Any combination of anchor elements, including friction-fit polyaxial pedicle screws 18, favored-angle pedicle screws 20, and/or laminar hooks 22 as described above may be used to secure the rods 12 to the cervical and/or thoracic vertebrae. When using the favored-angle pedicle screws 20 described above, the surgeon uses the visual indicator 194 to determine the direction of the biased angulation. This will enable the surgeon to quickly align the various pedicle screws and insert the spinal rod therein. Once the rod has been secured to the occipital plate 14 and pedicle screws, crosslink connectors 16 may then be employed to maintain the spinal rods 12 at a desired distance from one another.

While the invention is susceptible to various modifications and alternative forms, specific embodiments thereof have been shown by way of example in the drawings and are herein described in detail. It should be understood, however, that the description herein of specific embodiments is not intended to limit the invention to the particular forms disclosed, but on the contrary, the invention is to cover all modifications, equivalents, and alternatives falling within the spirit and scope of the invention as defined herein.

1. A spinal fixation system, comprising:
an occipital fixation member configured for placement against an occipital bone, said occipital fixation member having least one aperture for receiving an anchor element therethrough to anchor said occipital fixation member to bone and at least one side-loading clamp member dimensioned to receive a spinal rod therein;
at least two polyaxial bone screws, each polyaxial bone screw having an anchor member and a receiving member, the anchor member mating with the receiving member such that the anchor member exhibits a biased angulation, each polyaxial bone screw further comprising a visual indicator of the direction of the biased angulation;
first and second elongated spinal rods, each of the first and second spinal rods configured to extend between the occipital fixation member and one of the polyaxial bone screws; and
a crosslink connector configured to engage the first and second spinal rods and secure the first and second spinal rods in a desired spatial relationship.

2. The spinal fixation system of claim 1, wherein the occipital fixation member includes at least one of an occipital plate and an eyelet connector.

3-4. (canceled)

5. The spinal fixation system of claim 1, wherein the visual indicator includes at least one of color coding, raised surfaces, notches, detents, partial coloration, laser markings, and etchings.

6. A surgical fixation plate configured for placement against a bony segment, comprising:
a body portion having a shape suitable for placement against a bony segment, the body portion having a first lateral side, a second lateral side, and a medial axis bisecting the plate between the first and second lateral sides, the body portion having a first bone contacting surface and a second surface opposite the first surface, the body portion further having a plurality of openings extending between the first and second surfaces, each opening configured to receive an anchor element; and
at least two clamp members, one clamp member extending laterally from each of the first and second lateral sides, each clamp member having a rod receiving portion, each clamp member including an aperture extending therethrough in communication with the rod receiving portion, the aperture configured to receive a locking element to lock a spinal rod within the rod receiving portion, the aperture angularly offset relative to an axis extending through the clamp member and perpendicular to the first bone contacting surface.

7. (canceled)

8. The surgical fixation plate of claim 6, wherein the body portion includes at least one longitudinal groove cut into at least one of the first and second surfaces, the longitudinal groove providing a region along which the spinal fixation plate is bendable.

9. The surgical fixation plate of claim 6, wherein each of the at least two clamp members includes a first clamp portion comprising an extension of the first bone contacting surface and a second clamp portion comprising a curved element extending generally perpendicularly away from the second surface, the first and second clamp portions cooperating to form the rod receiving portion.

10. (canceled)

11. The surgical fixation plate of claim 10, wherein the aperture extends through the second clamp portion.

12. The surgical fixation plate of claim 6, wherein the rod receiving portion is configured to provide a snap-fit engagement with a spinal rod.

13. The surgical fixation plate of claim 6, wherein the angular offset is approximately 20 degrees.

14. The surgical fixation plate of claim 6, further comprising a visual indicator of the direction of the angular offset.

15. The surgical fixation plate of claim 14, wherein the visual indicator includes at least one of color coding, raised surfaces, notches, detents, partial coloration, laser markings, and etchings.

16-27. (canceled)

28. The spinal fixation system of claim 1, wherein the anchor member includes a proximal head, a distal tip, and an elongated shaft extending between the proximal head and distal tip, the head being at least partially spherical in shape to allow for multi-axial movement of the anchor member prior to insertion into bone, the elongated shaft being at least partially threaded to obtain purchase within a bone segment, and wherein the receiving member includes a proximal end, a distal end, and an axial bore extending through the receiving member from the proximal end to the distal end, the distal end comprising a first generally planar surface a second generally curved surface, the generally curved surface intersecting the axial bore such that the axial bore has a non-planar distal opening at the distal end, the axial bore further comprising a seat having a partially spherical surface for receiving the proximal head of the anchor member.

29. The spinal fixation system of claim 28, wherein the receiving member further includes first and second extensions extending between the proximal end and distal end, the first and second extensions separated by a generally U-shaped recess, the generally U-shaped recess dimensioned to receive at least a portion of a spinal rod.

30. The spinal fixation system of claim 29, further comprising a compression cap positioned between the first and second extensions, the compression cap having a first surface for contacting the head of the bone screw and a second surface opposite the first surface for contacting at least a portion of the spinal rod.

31. The spinal fixation system of claim 30, wherein the first and second extensions each include an at least partially threaded inner surface.

32. The spinal fixation system of claim 31, further comprising a threaded set screw configured to engage the at least partially threaded portions of the first and second extensions, the set screw including a rod-engaging surface.

33. (canceled)

34. The spinal fixation system of claim 28, wherein the second generally curved surface curves in a generally proximal direction.

35. The spinal fixation system of claim 34, wherein the distal opening is asymmetrical resulting in a zone of angulation having a maximum biased directional angulation and a minimum biased directional angulation.

36. The spinal fixation system of claim 35, wherein the maximum biased angulation comprises an angle of approximately 55 degrees.

37. The spinal fixation system of claim 35, wherein the minimum biased directional angulation comprises an angle of approximately 10 degrees.

38. The spinal fixation system of claim 35, wherein the visual indicator indicates the location of the maximum biased directional angulation.

39. (canceled)

40. A method for performing spinal fixation surgery, comprising the steps of:
(a) accessing surgical target site, the surgical target site comprising an occiput and at least one vertebra;
(b) providing a bone plate, the bone plate including:
a body portion having a shape suitable for placement against a bony segment, the body portion having a first lateral side, a second lateral side, and a medial axis bisecting the plate between the first and second lateral sides, the body portion having a first bone contacting surface and a second surface opposite the first surface, the body portion further having a plurality of openings extending between the first and second surfaces, each opening configured to receive an anchor element,
a clamp member extending laterally from one of the first and second lateral sides, the clamp member having a rod receiving portion and an aperture extending through the clamp member in communication with the rod receiving portion, the aperture configured to receive a locking element to lock a spinal rod within the rod receiving portion, the aperture angularly offset relative to an axis extending through the clamp member and perpendicular to the first bone contacting surface, and
a visual indicator of the direction of the angular offset;
(c) placing the bone plate against an occiput bone of a human skull such that the visual indicator of the direction of angular offset indicates that the angular offset is oriented in a cranial direction;
(d) inserting a plurality of anchor elements through the openings to secure the bone plate to the occiput bone;
(e) providing a polyaxial bone screw having an anchor member and a receiving member, the anchor member mating with the receiving member such that the anchor member exhibits a biased angulation, each polyaxial bone screw further comprising a visual indicator of the direction of the biased angulation,
(f) inserting the polyaxial bone screw into a bone such that the visual indicator of the direction of the biased angulation indicates that the biased angulation is oriented in a cranial direction; and
(g) inserting a spinal rod such that a first portion of the spinal rod is received within the clamp member of the bone plate and a second portion of the spinal rod is received within the receiving member of the polyaxial bone screw.

41. The method of claim 40, wherein the visual indicator of the direction of angular offset comprises at least one of color coding, raised surfaces, notches, detents, partial coloration, laser markings, and etchings.

42. The method of claim 40, wherein the visual indicator of the direction of biased angulation comprises at least one of color coding, raised surfaces, notches, detents, partial coloration, laser markings, and etchings.
